(19) United States
(12) Patent Application Publication
(10) Pub. No.: US 2006/0240536 A1
(43) Pub. Date: Oct. 26, 2006

(54) Fermentation broth extraction of K-252a

(76) Inventors: Sanjay R. Chemburkar, Gurnee, IL (US); Stephen S. Ulrey, Lake Bluff, IL (US); Julie J. Pruyne, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/406,687

(22) Filed: Apr. 19, 2006

Related U.S. Application Data
(60) Provisional application No. 60/673,006, filed on Apr. 20, 2005.

(51) Int. Cl. C12P 1/02

(52) U.S. Cl. 435/171; 435/41

(57) ABSTRACT

Methods for separating K-252a from the cell mass of its parent fermentation broth are disclosed.

This application claims priority to U.S. provisional Application Ser. No. 60/673,006, filed Apr. 19, 2005.

### FIELD OF THE INVENTION

This invention pertains to methods for separating K-252a from its parent fermentation broth.

### BACKGROUND OF THE INVENTION

The compound identified herein as K-252a is an intermediate in syntheses of compounds which are useful as medicaments. Large-scale production of K-252a, however, is compromised by its inability to be easily separated from the cell mass of its parent fermentation broth. There is therefore an existing need in the process and therapeutic arts for methods of separating K-252a from the cell mass of its parent fermentation broth.

### SUMMARY OF THE INVENTION

Accordingly, one embodiment of this invention pertains to a method for separating K-252a from the cell mass of its parent fermentation broth, said method comprising:
(a) providing a parent fermentation broth, said parent fermentation broth comprising water and cell mass, said cell mass having associated therewith intercellular or extracellular K-252a;
(b) mixing said parent fermentation broth and a solvent in which K-252a has solubility, and with which said parent fermentation broth forms a substantially separable emulsion, and isolating an extract, said extract comprising the solvent, water and K-252a; and
(c) repeating or not repeating step (b) with said solvent and isolating said K-252a.

Another embodiment pertains to a method for separating K-252a from the cell mass of its parent fermentation broth, said method comprising:
(a) providing a parent fermentation broth, said parent fermentation broth comprising water and cell mass, said cell mass having associated therewith intercellular or extracellular K-252a;
(b) mixing said parent fermentation broth and a solvent in which K-252a has solubility, and with which said parent fermentation broth forms a substantially separable emulsion, and isolating an extract, said extract comprising the solvent, water and K-252a; and
(c) repeating or not repeating step (b) with said isolated extract therefrom and isolating said K-252a.

Still another embodiment pertains to a method for separating K-252a from the cell mass of its parent fermentation broth, said method comprising:
(a) providing an isolated cell mass from a parent fermentation broth, said isolated cell mass having associated therewith water and intercellular or extracellular K-252a;
(b) mixing said cell mass and a solvent in which K-252a has solubility and isolating an extract, said extract comprising the solvent, water and K-252a; and
(c) repeating or not repeating step (b) with said solvent and isolating said K-252a.

Still another embodiment pertains to a method for separating K-252a from the cell mass of its parent fermentation broth, said method comprising:
(a) providing an isolated cell mass from a parent fermentation broth, said isolated cell mass having associated therewith water and intercellular or extracellular K-252a;
(b) mixing said cell mass and a solvent in which K-252a has solubility and isolating an extract, said extract comprising the solvent, water and K-252a; and
(c) repeating or not repeating step (b) with said isolated extract therefrom and isolating said K-252a.

Still another embodiment pertains to K-252a prepared by any of the foregoing methods.

### DETAILED DESCRIPTION OF THE INVENTION

The term “K-252a,” as used herein means the compound having formula (I)

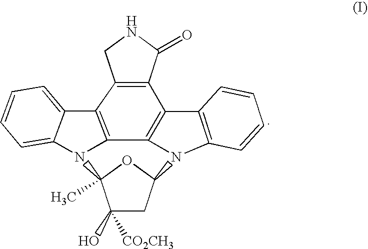

For the purposes of this invention K-252a is assigned the name methyl (1R,2S,4R)-2,4-(6,7,12,13-tetrahydro-5H-indolo[2,3-a]pyrrolo[3,4-c]carbazol-5-onediyl)-1-hydroxy-2-methyltetrahydrofuran carboxylate.

K-252a is a useful intermediate in syntheses of compounds having formula (II) and formula (III) 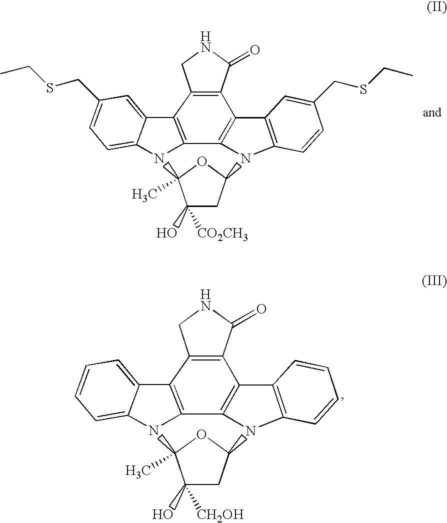
which are potentially useful for treating psorasis or acute myleogenous leukemia.

Parent fermentation broths have been found to contain between about 1 gram and 4 grams of K-252a per Kg. The difficulty in separating K-252a from the cell mass of its parent fermentation broth is reported in Biosci. Biotechnol. Biochem., 62(8), 1627-1629, 1998.

Without being limited by theory, it is believed that this difficulty is due, at least in part, to the formation of substantially inseparable emulsions which interfere with the formation of a visible interface layer. Accordingly, the phrase “substantially separable emulsion,” as used herein, means two or more than phases having a visible interface layer or layers, respectively. The term “interface layer,” as used herein, means the point in a mixture of two or more than two essentially immiscible solvents where the solvents meet.

The term “solvent,” as used herein means a liquid substance or a mixture of more than one liquid substance. Examples of solvents for the practice of this invention include, but are not limited to, acetone, ethanol, isopropanol, methanol, ethyl acetate, methyltetrahydrofuran (MTHF), methyl acetate, mixtures thereof and mixtures of the foregoing and water.

It is meant to be understood that, because many solvents contain impurities, the level of impurities in solvents for the practice of this invention, if present, are at a low enough concentration that they do not interfere with the intended use of the solvent in which they are present.

It is also meant to be understood that, for mixtures of essentially immiscible solvents, while one solvent may be somewhat soluble in another solvent, there is enough of a solubility difference between the solvents that a visible interface layer forms.

TABLE 1 shows the solubility of K-252a in various solvents at 22° C.

TABLE 1

| Solvent | Solubility (mg K-252a/g solvent) |
|---|---|
| acetone | 7.99 |
| methanol | 0.72 |
| MTHF | 27.99 |
| methyl acetate | >11.7 |
| 1% water/acetone | 9.36 |
| 10% water/acetone | 18.25 |
| 20% water/acetone | 17.78 |
| 30% water/acetone | 12.40 |
| 10% water/methanol | 0.54 |
| 20% water/methyl acetate | >17.7 |
| 10% water/MTHF (kinetic) | 104.07 |
| 10% water/MTHF (thermodynamic) | 31.97 |
| 5% acetone/methanol | 0.90 |
| 10% acetone/methanol | 1.22 |
| 50% acetone/methanol | 7.93 |
| 75% acetone/methanol | 18.59 |
| 10% MTHF/methanol | 1.20 |

Preferred solvents for the practice of this invention, when extracting a parent fermentation broth with one liquid substance with which the parent fermentation broth forms a substantially separable emulsion, include, but are not limited to methyltetrahydrofuran and methyl acetate.

Preferred solvents for the practice of this invention, when extracting a parent fermentation broth with a mixture of more than one liquid substance with which the parent fermentation broth forms a substantially separable emulsion, include, but are not limited to ethyl acetate or isopropyl acetate in combination with THF, methanol, ethanol or isopropanol.

During the processes of this invention, a step comprising substantially aggregating, isolating or removing cell mass may be used to facilitate the extraction, isolation or purification of K-252a. Aggregating, isolating or removing a cell mass may be accomplished using an ultrafiltration membrane or by centrifugating and decanting. If either of these techniques is used on a parent fermentation broth, an isolated cell mass having associated therewith some residual water and intercellular or extracellular K-252a can be obtained. An advantage of extracting an isolated cell mass instead of extracting a parent fermentation broth is that a broader range of solvents may be used with the former.

For example, single extraction of a parent fermentation broth with ethyl acetate, isopropyl acetate or THF having a broth/solvent ratio of about 1:≦1 caused an inseparable emulsion to form. However, ethyl acetate or isopropyl acetate in combination with THF, methanol, ethanol or isopropanol in the same broth/solvent ratio provided a substantially separable emulsion, as did methyl acetate alone with a broth/solvent ratio of about 1:2.

While two consecutive extractions of a parent fermentation broth with methyl acetate having a final broth/solvent ratio of about 1:8 gave nearly quantitative recovery of K-252a, in a more preferred process, the isolated extract from the previous extraction was used for a second extraction of the same parent fermentation broth to provide a similar result. Without being limited by theory, it is believed that water in the first extract increased the solubility of the K-252a and provided a more suitable second extraction medium.

Isolation of K-252a may be achieved by solvent removal with or without further purification. Purification of K-252a may be achieved by conventional means well-known in the art such as filtration, recrystallization, column chromatography or a combination thereof.

The following examples and tables are presented to provide what is believed to be the most useful and readily understood description of procedures and conceptual aspects of this invention.

### EXAMPLE 1

### Extraction With Methyl Acetate Percentages Based On Broth

Parent fermentation broth produced by Nocardiopsis sp. containing K-252a was mixed with methyl acetate (1:4 v/v), and a first extract was isolated. The broth was extracted again with fresh methyl acetate (1:4 v/v), and a second extract was isolated. Another parent fermentation broth containing K-252a was mixed with the first extract, and a third extract was isolated. The second parent fermentation broth was then extracted with the second extract, and a fourth extract was isolated. The four extracts were used to reextract each broth, combined, washed twice with brine and concentrated.

Note: extractions may be performed by mixing the broth and methyl acetate and isolating the extract after settling (gravity) or centrifuging.

The concentrate was dissolved in methyltetrahydrofuran (1.0:0.067 (w/w)), and the solution was treated with FILTROL® (1.0:0.0017 (w/w)), stirred at 70° C. for not less than 15 minutes, filtered and concentrated. A solution of the concentrate in methanol (1.0:0.03 (w/w)) was refluxed with for not less than 1 hour, cooled to 22° C., stirred for 4 hours and filtered. The filtrant was washed with methanol (1.0:0.01 (w/w)) and dried at 60° C. for 16 hours to provide between 88% and 94% of the estimated amount of K-252a.

### EXAMPLE 2

### Extraction with Methyltetrahydrofuran Percentages Based On Broth

Parent fermentation broth containing K-252a was centrifuged and decanted (after which the remaining cell mass may optionally be treated with water (1:2 w/w), stirred for 5 minutes, centrifuged and decanted). The remaining cell mass was extracted with methyltetrahydrofuran (1:1.25 (w/w)) for 5 minutes, centrifuged and decanted; and this procedure was repeated. The combined extracts were washed twice with brine (1:0.375 (w/w)), treated with FILTROL® filter aid (1:0.0025 (w/w)), stirred at 70° C. for 15 minutes, filtered hot and concentrated. A solution of the concentrate in methanol (1.0:0.03 (w/w)) was refluxed with for 1 hour, cooled to 22° C., stirred for 4 hours and filtered. The filtrant was washed with methanol (1.0:0.01 (w/w)) and dried at 60° C. for 16 hours to provide 100% the estimated amount of K-252a.

### EXAMPLE 3A

### Extraction With Acetone Percentages Based On Broth

Parent fermentation broth containing K-252a was centrifuged and decanted (after which the remaining cell mass may optionally be treated with water (1:2 w/w), stirred for 5 minutes, centrifuged and decanted). The remaining cell mass extracted with acetone (1:0.5 (w/w)) for 5 minutes, centrifuged and decanted; and this procedure was repeated. The combined extracts were treated with FILTROL® (1:0.002 (w/w)), stirred at 22° C. for 15 minutes, filtered, and concentrated to about two-tenths original volume. Remaining acetone was removed by adding methanol (1:0.05 (w/w)) and distilling to the original volume. The concentrate was treated with methanol (1:0.1 (w/w)), stirred at 70° C. for 1 hour then at 22° C. for 4 hours, filtered, washed with water or methanol (1:0.02 (w/w)), and air dried to provide a wet cake.

### EXAMPLE 3B

### Percentages Based On Wet Cake

The wet cake was treated with 25% (w/w) methanol/acetone (1:20 (w/w)) and FILTROL® (1:0.5 (w/w)), and the slurry was refluxed for 1 hour, filtered hot, washed with 25% (w/w) methanol/acetone (1:5 (w/w)), concentrated to half-volume, treated with methanol (1:10 (w/w)), refluxed for 1 hour, cooled to 22° C., stirred 4 hours, and filtered. The filtrant was washed with methanol (1:5 (w/w)) and dried under vacuum at 60° C.-65° C. for 16 hours to provide 95% the estimated amount of K-252a.

### EXAMPLE 4

### Extraction With Ethyl Acetate/Ethanol Mixtures

Parent fermentation broth containing K-252a (100 mL; broth potency:1.42 mg/mL) was extracted with ethyl acetate (10 mL)/ethanol (50 mL) then twice with ethyl acetate (70 mL)/ethanol (10 mL) to provide extracts containing 82%, 15% and 3%, respectively, of the K-252a (HPLC). The extracts were combined, filtered through a hyflo pad, and concentrated to near-dryness. The concentrate was treated with methanol (6 mL), cooled to room temperature, and filtered. The filtrant was washed with minimal methanol and dried under vacuum at 60° C. to provide K-252a (136 mg).

### EXAMPLE 5

### Extraction With Isopropyl Acetate/Isopropanol

Parent fermentation broth containing K-252a (100 mL; broth potency: 1.42 mg/mL) was extracted with isopropyl acetate (90 mL)/isopropanol (50 mL) then twice with isopropyl acetate (80 mL)/isopropanol (20 mL) to provide extracts containing 78%, 11% and 11%, respectively, of the K-252a (HPLC). The extracts were combined, filtered through a hyflo pad, and concentrated to near-dryness. The residue was dissolved in tetrahydrofuran (20 mL), filtered through a hyflo pad, and concentrated to near-dryness. The concentrate was treated with methanol (5 mL), cooled to room temperature and filtered. The filtrate was washed with methanol (4 mL) and dried under vacuum at 60° C. to provide K-252a (147 mg).

TABLE 2, TABLE 3 and TABLE 4 summarize results of other extraction processes.

TABLE 2
In this process, the solvent used was a mixture of methyl acetate
(MeOAc), methyltetrahydrofuran (MTHF) and methanol.

| | | | |
|---|---|---|---|
| Broth Amt.: | 0.2 | Kg | 3.0 |
| MeOAc (g/g broth): | 0.8 | | 0.8 |
| MTHF (g/g broth): | 0.06 | | 0.06 |
| Methanol (g/g broth): | 0.04 | | 0.04 |
| Isolated K-252a Amt.: | 0.3 | g | 4.3 |

TABLE 3
In this process, the solvent used was a mixture of
methyltetrahydrofuran (MTHF) and methanol.

| | |
|---|---|
| Broth Amt.: | 0.2 Kg |
| MTHF (g/g broth): | 0.5 |
| Methanol (g/g broth): | 0.04 |
| Isolated K-252a Amt.: | 0.37 g |

TABLE 4
In this process, the solvent used was a mixture of acetone and methanol.

| | | | |
|---|---|---|---|
| Broth Amt.: | 1 | Kg | 1 |
| Acetone (g/g broth): | 1 | | 1 |
| Methanol (g/g broth): | 0.25 | | 0.25 |
| Isolated K-252a Amt.: | 0.96 | g | 3.7 |

The foregoing is meant to be illustrative of the invention and not intended to limit it to the disclosed embodiments. Variations and changes obvious to one skilled in the art are intended to be within the scope and nature of the invention as defined in the claims.

We claim:

1. A method for separating K-252a from the cell mass of its parent fermentation broth, said method comprising:
(a) providing a parent fermentation broth, said parent fermentation broth comprising water and cell mass, said cell mass having associated therewith intercellular or extracellular K-252a;
(b) mixing said parent fermentation broth and a solvent in which K-252a has solubility, and with which said parent fermentation broth forms a substantially separable emulsion, and isolating an extract, said extract comprising the solvent, water and K-252a; and
(c) repeating or not repeating step (b) with said solvent and isolating said K-252a.

2. The method of claim 1, wherein the solvent is acetone, methyl acetate, methyltetrafuran or a mixture thereof.

3. The method of claim 2, wherein the solvent is methyl acetate.

4. The method of claim 2, wherein the solvent is methyl tetrahydrofuran.

5. The method of claim 2, wherein the solvent is acetone.

6. A method for separating K-252a from the cell mass of its parent fermentation broth, said method comprising:
(a) providing a parent fermentation broth, said parent fermentation broth comprising water and cell mass, said cell mass having associated therewith intercellular or extracellular K-252a;
(b) mixing said parent fermentation broth and methyl acetate and isolating an extract, said extract comprising the methyl acetate, water and K-252a; and
(c) repeating or not repeating step (b) with said isolated extract therefrom and isolating said K-252a.

7. A method for separating K-252a from the cell mass of its parent fermentation broth, said method comprising:
(a) providing a parent fermentation broth, said parent fermentation broth comprising water and cell mass, said cell mass having associated therewith intercellular or extracellular K-252a;
(b) mixing said parent fermentation broth and methyl tetrahydrofuran and isolating an extract, said extract comprising the methyl acetate, water and K-252a; and
(c) repeating or not repeating step (b) with said isolated extract therefrom and isolating said K-252a.

8. A method for separating K-252a from the cell mass of its parent fermentation broth, said method comprising:
(a) providing a parent fermentation broth, said parent fermentation broth comprising water and cell mass, said cell mass having associated therewith intercellular or extracellular K-252a;
(b) mixing said parent fermentation broth and acetone and isolating an extract, said extract comprising the methyl acetate, water and K-252a; and
(c) repeating or not repeating step (b) with said isolated extract therefrom and isolating said K-252a.

9. A method for separating K-252a from the cell mass of its parent fermentation broth, said method comprising:
(a) providing an isolated cell mass from a parent fermentation broth, said isolated cell mass having associated therewith water and intercellular or extracellular K-252a;
(b) mixing said cell mass and a solvent in which K-252a has solubility and isolating an extract, said extract comprising the solvent, water and K-252a; and
(c) repeating or not repeating step (b) with said solvent and isolating said K-252a.

10. A method for separating K-252a from the cell mass of its parent fermentation broth, said method comprising:
(a) providing an isolated cell mass from a parent fermentation broth, said isolated cell mass having associated therewith water and intercellular or extracellular K-252a;
(b) mixing said cell mass and a solvent in which K-252a has solubility and isolating an extract, said extract comprising the solvent, water and K-252a; and
(c) repeating or not repeating step (b) with said isolated extract therefrom and isolating said K-252a.
